(19) United States
(12) Patent Application Publication
(10) Pub. No.: US 2016/0338870 A1
(43) Pub. Date: Nov. 24, 2016

(54) Anti-Snoring Device

(71) Applicant: Graeme Francis Walton, Main Beach (AU)

(72) Inventor: Graeme Francis Walton, Main Beach (AU)

(21) Appl. No.: 15/112,864

(22) PCT Filed: Jan. 21, 2015
(86) PCT No.: PCT/AU2015/050019

§ 371 Date: Jul. 20, 2016

(30) Foreign Application Priority Data
Jan. 24, 2014 (AU) 2014900219

(51) Int. Cl. A61F 5/56; A61F 5/37

(52) U.S. Cl. CPC A61F 5/56; A61F 5/37

(57) ABSTRACT

The present invention relates in general to a device to assist in the prevention of snoring. The device comprising: a membrane shaped as an elongate oval having opposed sides and opposed arcuate ends, the membrane having a front and a rear face; a hypoallergenic adhesive applied uniformly to the rear face of said membrane, and a backing sheet which covers the adhesive on the back of said membrane; at least one tab formed at one of the arcuate ends of the membrane, such that the at least one tab allows the membrane to be easily manipulated between open and closed positions; and wherein the device is sized and configured to substantially cover a user's closed lips and an area of skin adjacent to each lip to hold the lips in a closed position and therefore provide a good seal of the device to the area around the mouth of the user.

### FIELD OF THE INVENTION

The present invention relates in general to a device for the prevention of snoring. In particular, the present invention relates to devices designed to be used by human beings for the prevention of mouth breathing and thus be worn by a sleeping person. The invention also provides a device which aids in keeping the bottom or lower jaw in place to keep the lips closed to ensure that the user's tongue remains forward keeping the airways open and allowing them to breathe through their nose.

### BACKGROUND OF THE INVENTION

It should be noted that reference to the prior art herein is not to be taken as an acknowledgement that such prior art constitutes common general knowledge in the art.

It is generally accepted that snoring results from the vibration of respiratory structures and the resulting sound, due to obstructed air movement during breathing while sleeping. In some cases the sound may be soft, but in other cases, it can be loud and unpleasant. The snoring sound is largely the result of vibrations of the soft palate and uvula when air is breathed in through the mouth.

Existing devices for prevention of snoring or air intake through the mouth require the insertion of such devices in the mouth or in the nose, or require the use of straps tied around the head or neck so as to hold such devices in place. Such devices are uncomfortable and are not widely accepted. While existing devices may be successful in arresting or otherwise correcting the habit of snoring, none of the devices is easily manipulable by the user.

Clearly it would be advantageous if a device for preventing snoring could be devised that helped to at least ameliorate some of the shortcomings described above. In particular, it would be beneficial to create an anti-snoring device which is either fitted easily over the lips of the user or around the lips to close the bottom jaw which inhibits mouth breathing and thus could be easily worn by a sleeping person.

### SUMMARY OF THE INVENTION

The present invention provides a specially designed patch that covers your mouth and supports the lower jaw to keep it closed to stop snoring during sleep. The device is simple, comfortable and easy to use. The device can be placed either over your mouth or around your lips around the lower jaw to hold the jaw in place so that your tongue stays forward keeping your airways open allowing you to breathe naturally through your nose.

In accordance with a first aspect, the present invention provides an anti-snoring device comprising: a membrane shaped as an elongate oval having opposed sides and opposed arcuate ends, the membrane having a front and a rear face; a hypoallergenic adhesive applied uniformly to the rear face of said membrane, and a backing sheet which covers the adhesive on the back of said membrane; at least one tab formed at one of the arcuate ends of the membrane, such that the at least one tab allows the membrane to be easily manipulated between open and closed positions and wherein the device is sized and configured to substantially cover a user's closed lips and an area of skin adjacent to each lip to hold the lips in a closed position.

Preferably, the opposed sides may comprise parallel linear sides. Alternatively the opposed sides may comprise opposed curved sides. Further alternatively, the opposed sides may comprise continuously convex curved opposed sides.

Preferably, the membrane may comprise a sheet of flexible, tear-resistant plastic or woven fabric material. The tear-resistant material may be a non-woven fabric. Preferably, the membrane may be manufactured from cotton, plastic or nylon, and the hypoallergenic adhesive is an epoxy resin.

Preferably, the sheet of flexible material may have two tabs, each tab formed at each arcuate end of the membrane. The tab or tabs on their rear surface may be free from any hypoallergenic adhesive. The hypoallergenic adhesive may be releasable and designed to be opened and closed without losing the ability to sufficiently hold the lips in the closed position.

Preferably, the membrane may be a transparent, multi-coloured or skin coloured tear resistant material. The membrane may have a design or picture(s) on one of the opposed sides of the tear resistant material.

Preferably, the parallel linear sides of the membrane may be separated by a distance which equates to a distance measured to substantially cover a user's closed lips and an area of skin adjacent to each lip. Preferably, the membrane may be supplied in at least five sizes ranging from children to adults, the at least five sizes equate to five different heights which cover a large number of the user's lip height when closed plus the distance measured to substantially cover the user's closed lips and an area of skin adjacent to each lip. The shape of the membrane will allow for a variety of different shaped and sized lips.

Preferably, the opposed linear sides of said device may be continuously convexly curved to generally conform to the contour of the user's upper and lower lips respectively and an area of skin adjacent to each lip.

Preferably, said adhesive may be applied to an area located adjacent the outer edges of said membrane.

Preferably, the anti-snoring device may further comprise an aperture located substantially in the centre of the device to provide an air vent for the device.

Preferably, the anti-snoring device may further comprise a curved section located in one of the opposed sides to allow the device to be easily located within the contour or philtrum in between the top lip and the bottom of the nose of the user.

In accordance with a further aspect, the present invention provides an anti-snoring device for applying over the lips of a user comprising: a flexible, elongate membrane having a perimeter, a generally uniform thickness and opposed faces; a hypoallergenic adhesive applied uniformly to one face of said membrane, and a backing sheet which covers the adhesive; at least one tab extending from an end of the membrane, such that the at least one tab allows the membrane to be easily manipulated between open and closed positions; and wherein the device is sized and configured to substantially cover the user's closed lips and an area of skin adjacent to each lip to hold the lips in a closed position to maintain a good seal of the device to the user.

Preferably, said membrane may be shaped as an elongate oval having parallel opposed linear sides and opposed arcuate ends. Alternatively, said membrane may be shaped as an elongate oval having curved opposed sides and opposed arcuate ends.

Preferably, the at least one tab may be free from adhesive to allow the device to be easily opened and closed.

In accordance with a still further aspect, the present invention provides an anti-snoring device for applying around the lower lip of a user comprising: a flexible, elongate membrane having a perimeter, a generally uniform thickness and opposed faces; a hypoallergenic adhesive applied uniformly to one face of said membrane, and a backing sheet which covers the adhesive; a plurality of tabs extending around the perimeter of the membrane, such that the tabs allows the membrane to be easily manipulated between open and closed positions; and wherein the device is sized and configured to substantially fit around the user's closed lower lip and an area of skin adjacent either end of the lips to hold the lower jaw in a closed position to inhibit mouth breathing.

Preferably, the flexible elongate membrane may have a upper edge and a lower edge extending around the perimeter, wherein the lower edge conforms to the shape of the lower edge of the face of the user, including the area defined by the chin boss and extending to an area on either end of the lips to secure the lower jaw closed to prevent mouth breathing.

Preferably, the flexible elongate membrane may have an upper edge and a lower edge extending around the perimeter, wherein the lower edge conforms to the shape of the lower edge of the face of the user, including the area defined by the chin boss and extending to an area spaced apart from either end of the lips to secure the lower jaw closed to prevent mouth breathing.

In accordance with a still further aspect, the present invention provides a method of using an anti-snoring device comprising the steps of: (a) providing a membrane shaped as an elongate oval having opposed sides and opposed arcuate ends, the membrane having a front and a rear face with adhesive applied to the rear face; (b) removing a backing sheet to expose the adhesive layer; (c) applying the membrane to a user's face; and (c) using tabs located on the device to open and close the device as desired, wherein the tabs on their rear surface are free from any adhesive.

Preferably, the method may comprise any one of the features as claimed in the device of any of the previous embodiments.

### BRIEF DESCRIPTION OF THE DRAWINGS

The present invention will be understood more fully from the detailed description given hereinafter and from the accompanying drawings of the preferred embodiment of the present invention, which, however, should not be taken to be limitative to the invention, but are for explanation and understanding only.

FIG. 1 is a plan view of an anti-snoring device in accordance with an embodiment of the present invention;

FIG. 2 is a bottom view of the anti-snoring device of FIG. 1 showing distribution of adhesive around the central section of the device;

FIG. 3 illustrates a frontal view of the anti-snoring device when applied to cover a user's mouth;

FIG. 4 illustrates a side view of the anti-snoring device of FIG. 3 when applied to cover the user's mouth;

FIG. 5 illustrates a frontal view of the anti-snoring device in use showing the easy manipulation of the device;

FIG. 6 illustrates a frontal view of the anti-snoring device in use showing the easy manipulation of the device by a user when taking a drink;

FIG. 7 is a plan view an anti-snoring device in accordance with a further embodiment of the present invention;

FIG. 8 is a bottom view of the anti-snoring device of FIG. 7 showing distribution of adhesive around the central section of the device;

FIG. 9 illustrates a front view of an anti-snoring device in accordance with a further embodiment of the present invention;

FIG. 10 illustrates a front view of an anti-snoring device in accordance with a further embodiment of the present invention;

FIGS. 11 and 12 show front and rear views of an anti-snoring device in accordance with a further embodiment of the present invention; and

FIGS. 13 and 14 show front and rear views of an anti-snoring device in accordance with a further embodiment of the present invention.

### DETAILED DESCRIPTION OF THE INVENTION

The following description, given by way of example only, is described in order to provide a more precise understanding of the subject matter of a preferred embodiment or embodiments.

Snoring results from the vibration of respiratory structures and the resulting sound, due to obstructed air movement during breathing while sleeping. In some cases the sound may be soft, but in other cases, it can be loud and unpleasant. The snoring sound is largely the result of vibrations of the soft palate and uvula when air is breathed in through the mouth. In order to prevent or inhibit snoring the present invention has been devised to create an anti-snoring device which is fitted easily around and over the lips of the user to inhibit mouth breathing and thus can be easily worn by a sleeping person.

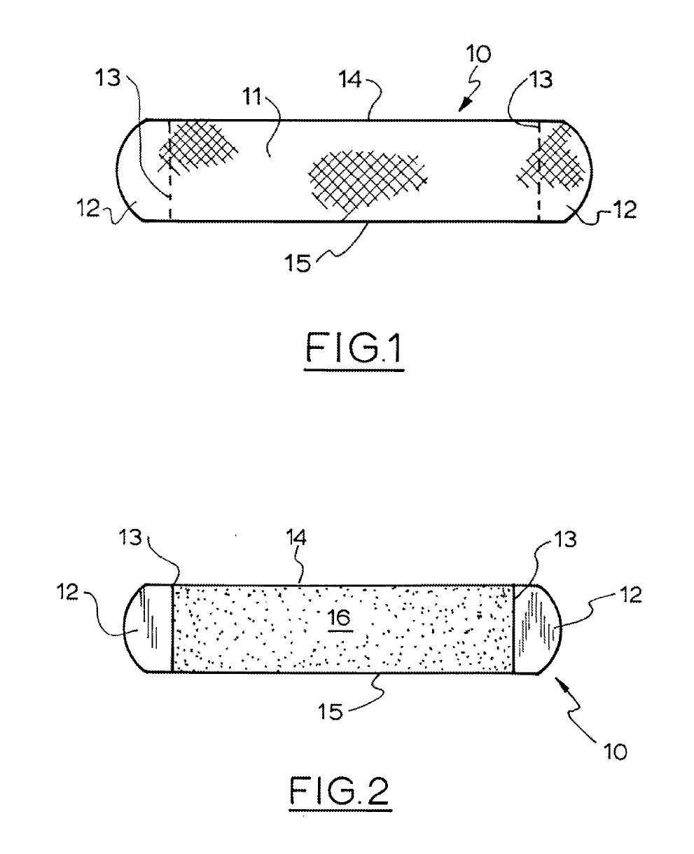

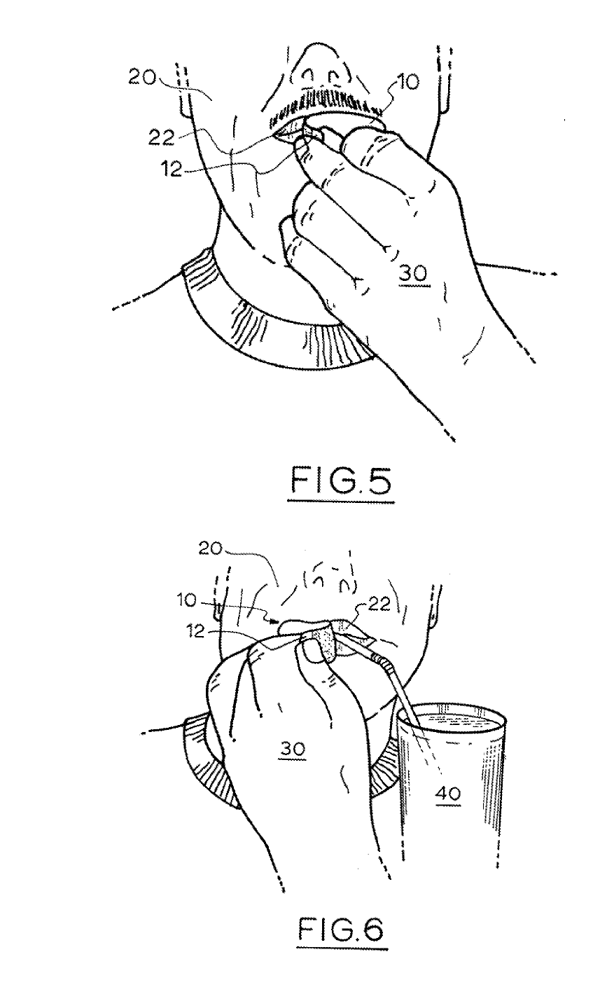

FIGS. 1 and 2 illustrate an embodiment of an anti-snoring device 10 with FIG. 1 showing the anti-snoring device as viewed from the front and FIG. 2 shows the anti-snoring device as viewed from behind. The anti-snoring device 10 consists of a sheet of material or membrane 10 shaped as an elongate oval having parallel opposed linear sides 14, 15 and opposed arcuate ends 12. The sheet of material or membrane 10 having a front face 11 and a rear face 16. The opposed parallel sides 14, 15 being separated by a distance which equates to the average height across the lips 22 of user 20. In order to cover as many user's lip heights as possible the present invention extends to a sheet or membrane 10 which is manufactured in approximately five different height variations. The five different height variations have been designed so that they will cover the many different lip heights as possible. This also includes any user from children up to and including adults with different height variations. The opposed arcuate ends or tabs 12 are used to enable the user to be able to open and close the anti-snoring device 10. The device 10 has been designed so that it can be resealable on the lips 22 and the area around the lips 22 of the user 20. This will be further described below in relation to FIGS. 5 and 6.

Typically these devices 10 are manufactured from a sterile sheet of flexible, tear-resistant paper or plastic or from any non-woven fabric material. The membrane or sheet 10 is thin and flexible with sufficient strength to withstand tearing when in use. Alternatively the tear resistant fabric may be a woven material. The woven material or woven fabric is a textile formed by weaving, for example cotton. A non-woven fabric is typically a fabric-like material made from long fibres, bonded together by chemical, mechanical, heat or solvent treatment. For example, paper is a non-woven fabric and is broadly defined as a sheet or web structure bonded together by entangling fiber or filaments (and by perforating films) mechanically, thermally or chemically. As a further alternative the sheet or membrane 10 may be fiber reinforced for extra strength and tear-resistance.

The front face 11 of the device 10 may be transparent, multi-coloured or skin coloured and may be waterproof. The front face 11 of the device 10 may include a graphic, drawing, logo or photo on the front face 11. For example, a device 10 for a child may include a cartoon character or some other child relevant graphic. Likewise the front face 11 may simply consist of multi-colours, for example the colours of a rainbow. The rear face 16 firstly has a layer of hypoallergenic adhesive, i.e., the side to be applied to area around the lips 22 and over the user's lips 22, and a strippable protective backing sheet (not shown) covering the adhesive back face 16. As illustrated in FIG. 2 the tab or arcuate ends 12 are free from any adhesive. The adhesive is applied on the rear face 16 in the area located between parallel opposed linear sides 14, 15 and a line 13 which delimits or indicates the boundary or start of the tabs 12 which are located on either end of the anti-snoring device 10.

An adhesive, preferably a sterile, pressure-sensitive, hypoallergenic adhesive is applied as a film on the back face 16 of the membrane 10. As described above the adhesive is applied uniformly over the back surface 16 with the exception of the area in which the end tabs 12 are located. Alternatively the adhesive can be applied over only a portion of the sheet of material, for example, a portion which lies adjacent to and around the edges 14, 15 and the lines 13 which show the start of the tabs 12 of the membrane or sheet of thin material 10. The adhesive is of such a character that it is non-drying, is capable of remaining in a tacky state, and yet can be readily removed from a user's skin without injury to the user. Typically and as widely used silicone adhesives, such as those used for adhesive bandage strips, may be used in the present invention.

During manufacture, the adhesive backing on the rear face 16 is covered by a strippable protective backing sheet. The backing sheet, preferably made of paper or plastic, can be coated on its inner face, i.e., the face to be attached to the adhesive, so that the device can easily be peeled off the backing sheet and so that very little adhesive will come off on the backing sheet. The backing sheet is of a size large enough to cover the rear face 16 to which the adhesive is applied. The paper or plastic backing sheet covers the area to which adhesive has been applied on the rear face 16 of the device 10. However as a further alternative the backing sheet can be made in the same size and shape as the anti-snoring device 10. In this alternative the backing sheet may be split to facilitate easy removal of the backing sheet when the device 10 is required for use.

Further and by way of example only, the adhesive may be found naturally or produced synthetically and by making the adhesive hypoallergenic, simply meaning “below normal” or “slightly” allergenic. The term hypoallergenic is used to describe items that cause or are reduced to cause fewer allergic reactions. It should therefore be understood that any type of material and any type of adhesive may be used for the above device 10 provided the material is substantially tear resistant and flexible to provide a seal around and over the lips 22 of the user 20 to inhibit mouth breathing and thus stop or substantially reduce the chances of the user snoring.

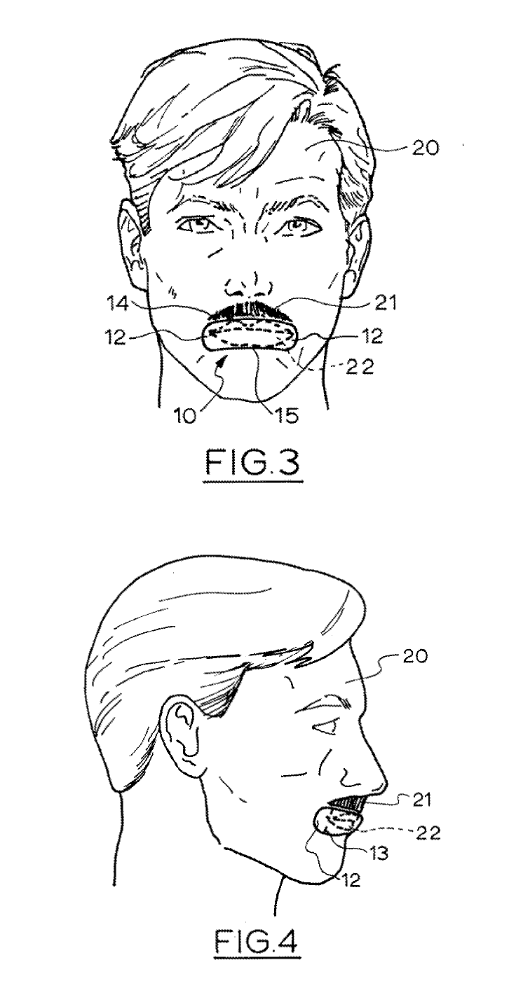

The present invention has been designed to largely cover the area around and adjacent the lips and including the lips 22 of a user 20. As illustrated in FIGS. 3 and 4 the sheet or membrane 10 has been designed to cover the user's lips 22 and the area adjacent either side and either end of the lips 22. In use, the parallel opposed linear side 14 lies along and just below the user's moustache 21, or if the user does not have a moustache along a straight line which runs just above the top lip of the user 20. The lower linear side 15 lies just below a line running across the chin of the user and just below the user's lower lip.

The size of the device is about two centimetres high and eight centimetres wide, as viewed in place over the lips 22 of a user 20. It is expected that this device 10 can be manufactured in smaller or larger sizes to accommodate extreme differences in a user's lip size, as mentioned above the sheet or membrane 10 may be manufactured in five different sizes. The different sizes designed to substantially cover any user from children up to and including adults. Although the abovementioned device of two centimetres by eight centimetres size should be satisfactory for the vast majority of users 20.

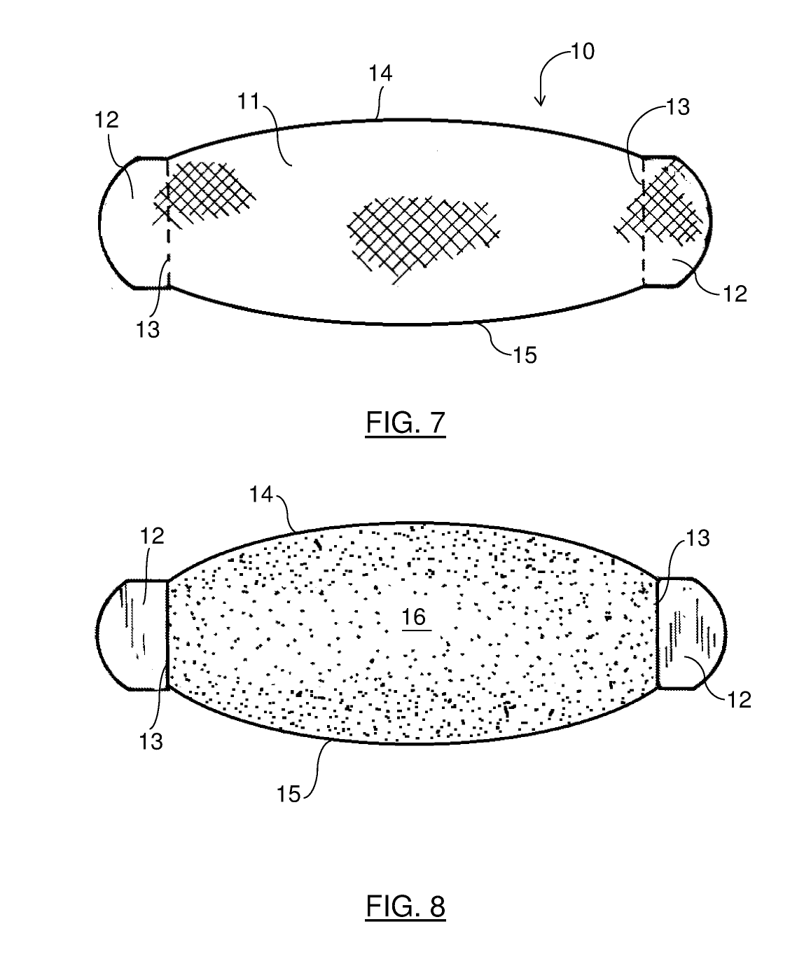

Alternatively and as illustrated in FIGS. 7 and 8 the device is about four to five centimetres high in the centre of the device 10 and narrows down to around two to three centimetres at the tab ends 12 and approximately ten centimetres wide, as viewed in place over the lips 22 and adjacent skin above and below the lips 22 of a user 20. This provides a better seal of the device 10 and aids to keep the device 10 in place so that it will not blow-off during sleep.

In an alternative embodiment (not shown) the sheet or membrane 10 may have opposing sides which are continuously convexly curved to generally conform to the contour of the user's upper and lower lips respectively. That is the sides 14, 15 of the device are largely designed to mimic the shape of the upper and lower lips 22 of a user 20.

FIGS. 5 and 6 illustrate the present invention in use and show how the sheet or membrane 10 can be easily open and closed to perform simple tasks which may eventuate while the user 20 is wearing the anti-snoring device 10. For example FIG. 5 show a user 20 using their hand and fingers 30 to manipulate a tab 12 to open an end of the device 10 and allow the user to talk to their partner in bed. Likewise FIG. 6 illustrates the user 20 using their hand and fingers 30 to remove one side 12 of the device 10 to allow the user to take a drink 40 while in bed. The present invention has been designed so that the adhesive applied to the back 16 of the device 10 can be opened and closed a number of times prior to the device 10 losing its ability to seal the lips 22 of the user and therefore not being able to prevent mount breathing in order to at least inhibit or prevent snoring.

Immediately prior to use of the device 10, the mouth and nose area of the user 20 should be cleansed with an oil-removing substance, for example, soap and water. The user's nasal passage must be cleared for breathing, and the mouth should be in a closed position. The device 10 is then peeled from its protective backing sheet and fitted over the user's lips 22 so that the adhesive backing is against the area around the user's lips 22 such that the device 10 completely covers the user's lips 22. As shown in FIGS. 5 and 6, the tabs 12 may be used to open and close the device 10. If for example the protective backing sheet is provided with a slit, the device 10 can first be fitted to the user's lips 22 and held in position by one tab 12 and then the other of the backing sheet is removed. Any minor discomfort, for example, as caused by an improper fit or because the user is not comfortable having a sheet or membrane over the user's lips can be ameliorated by removing and reapplying the device several times.

FIGS. 7 and 8 illustrate a further embodiment of the present invention in which the opposed sides 14, 15 are curved in shape so that the device covers the lips 22 of the user 20 but also a small amount of skin located adjacent both the top and bottom lips 22. This will aid in keeping the lower jaw in a closed position to inhibit or prevent the mouth breathing associated with snoring.

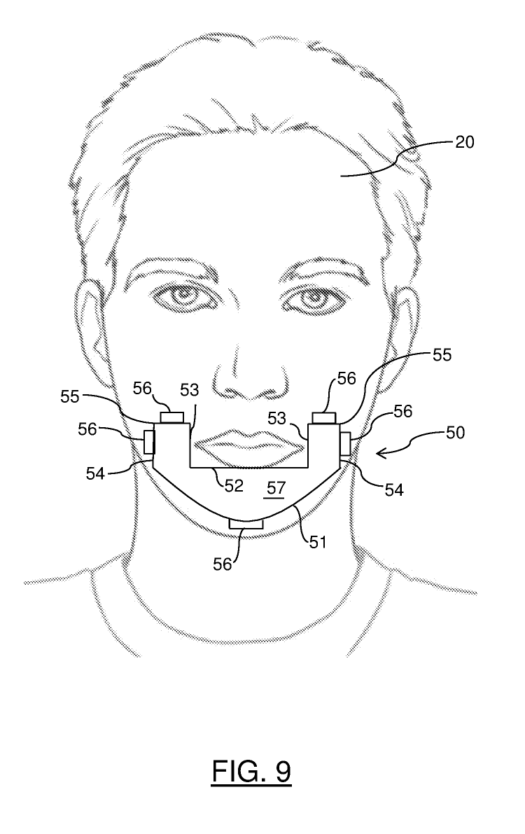

FIG. 9 shows a further embodiment of the anti-snoring device in accordance with the present invention. This embodiment has been designed in order to provide an anti-snoring device which assists to keep the lower jaw in place to keep the airways open. The device 50 is designed to fit around the lips of a user 20 to keep the jaw and lips in a closed position. The device 50 comprises top sides 52, 53 which are located around the mouth, top edges 55 and outer edges 54. The bottom edge 51 substantially conforms to the shape of the device 50 to the shape of the users 20 jaw and joins to the bottom of the outer edges 54. The anti-snoring device 50 consists of a sheet of material or membrane shaped to fit around the mouth of the user 20. The sheet of material or membrane having a front face 57 and a rear face.

As described above the device 50 is typically manufactured from a sterile or non-sterile sheet of flexible, tear-resistant paper or plastic or from any non-woven or woven fabric material. The membrane or sheet is thin and flexible with sufficient strength to withstand tearing when in use. The front face 57 of the device 50 may be transparent or skin coloured and may be waterproof. The rear face (not shown) firstly has a layer of hypoallergenic adhesive, i.e., the side to be applied to the user's face 20, and a strippable protective backing sheet (not shown) covering the adhesive back face. A number of tabs 56 are located around the device 50 the tab ends 56 are free from any adhesive. The tabs 56 allow the user 20 to easily manipulate the device 50 between open and closed positions. The adhesive is applied on the rear face in the area limited between sides 51, 52, 53, 54 and 55 of the anti-snoring device 50.

Like above the adhesive, preferably a sterile or non-sterile, pressure-sensitive, hypoallergenic adhesive is applied as a film on the back face of the device 50. The adhesive is applied uniformly over the back surface with the exception of the area in which the tabs 56 are located. The adhesive is of such a character that it is non-drying, is capable of remaining in a tacky state, and yet can be readily removed from a user's skin without injury to the user. Typically and as widely used silicone adhesives, such as those used for adhesive bandage strips, may be used in the present invention.

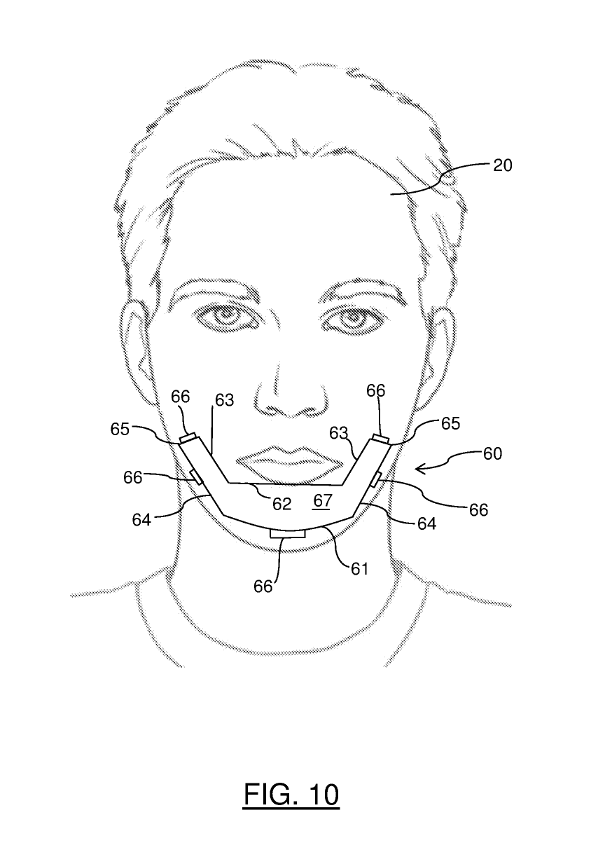

FIG. 10 shows a further embodiment of the anti-snoring device in accordance with the present invention. The device 60 is similar to the device 50 described above with a few differences designed to avoid any facial hair which may be on the users 20 top lip. This embodiment has been designed in order to provide an anti-snoring device which assists to keep the lower jaw in place to keep the airways open as described above for device 50. The device 60 comprises top sides 62, 63 which are located around the mouth, top edges 65 and outer edges 54. The bottom edge 51 substantially conforms to the shape of the device 50 to the shape of the users 20 jaw and joins to the bottom of the outer edges 64. The anti-snoring device 60 consists of a sheet of material or membrane shaped to fit around the mouth of the user 20. The sheets of material or membrane having a front face 67 and a rear face (not shown). The device 60 also has a number of tabs 66 located around the device 60 the tab ends 66 are free from any adhesive. The tabs 66 allow the user 20 to easily manipulate the device 60 between open and closed positions. The adhesive is applied on the rear face in the area limited between sides 61, 62, 63, 64 and 65 of the anti-snoring device 60. With the exception that the sides 63 are designed to locate the device 60 away from the top side of the lips it is designed in the same way as the device 50.

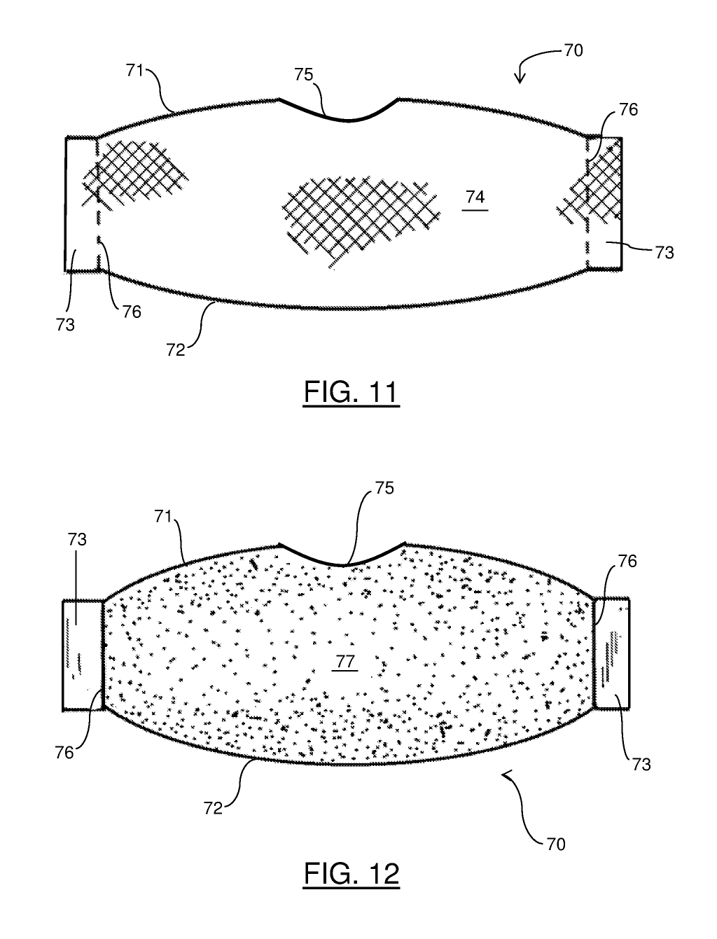

FIGS. 11 and 12 show front and rear views of a device 70 which is substantially the same as the device 10 described in FIGS. 7 and 8. The only differences being the top edge 71 of the device 70 and that of the device 10 described above is the indentation or curved section 75 which aids in attaching the device 70 to the contour or philtrum in the top lip of the user. The philtrum or medial cleft is a vertical groove in the middle area of the upper lip, extending from the nose to the upper lip.

The device 70 has opposed arcuate sides 71, 72 with a front face 74 and a rear face 77. The device 70 also has tabs 73 located at either end of the device 70 the tab ends 73 are free from any adhesive. The adhesive is limited to the area located between the sides 71, 72 and the lines 76 located at either end of the device 70. The tabs 73 allow the user to easily manipulate the device 70 between open and closed positions and removal from the face.

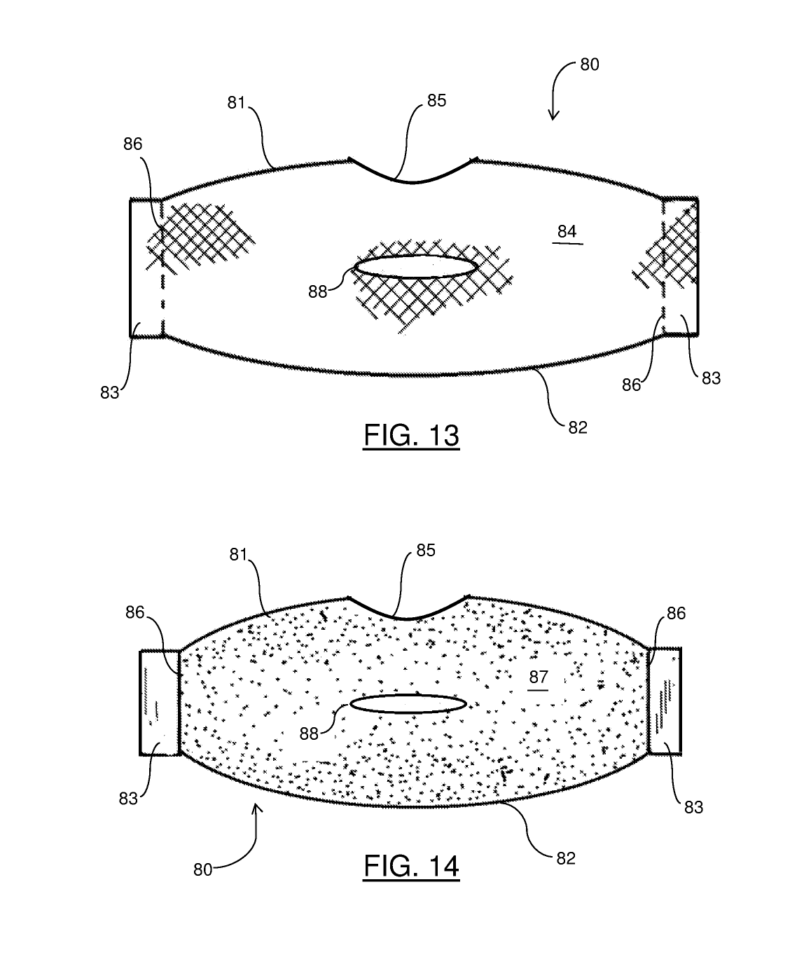

FIGS. 13 and 14 shows a further embodiment of the present invention in which the device 80 has an aperture 88 located substantially in the centre of the device 80. The aperture 88 provides an air vent which allows the user who may need to cough, sneeze or take that extra breath through the mouth if needed. The device 80 still keeps the lower jaw closed in order to inhibit or prevent snoring.

The device 80 has a top arcuate side 81 with a curved section 85 which aids in attaching the device 80 to the contour or philtrum in the top lip of the user. A bottom arcuate side 82 joins the opposite ends or tabs 83 to the top side 81. The device 80 has a front surface 84 and a rear surface 84 to which the adhesive is placed on. The membrane or sheet is thin and flexible with sufficient strength to withstand tearing when in use. The front face 84 of the device 80 may be transparent, multi-coloured or skin coloured and may be waterproof. The rear face 87 firstly has a layer of hypoallergenic adhesive, i.e., the side to be applied to the user's face, and a strippable protective backing sheet (not shown) covering the adhesive back face 87.

The device 80 also has tabs 83 located at either end of the device 80 the tab ends 83 are free from any adhesive. The adhesive is limited to the area located between the sides 81, 82 and the lines 86 located at either end of the device 80. The tabs 83 allow the user to easily manipulate the device 80 between open and closed positions.

Optionally, after manufacture, the device can be placed in a suitable package and be sterilized in a conventional manner for sanitation purposes. As a further option a number of the devices can be supplied in a container and grouped dependent upon the respective height and width of the lips and therefore the size of the device.

While a number of embodiments have been described all devices are manufactured with a membrane or sheet which is thin and flexible with sufficient strength to withstand tearing when in use. The front faces may be transparent, multi-coloured or skin coloured and may be waterproof. The front face of the anti-snoring device may include a graphic, drawing, logo or photo on the front face. Typically these devices are manufactured from a sterile sheet of flexible, tear-resistant paper or plastic or from any non-woven fabric material. Alternatively the tear resistant fabric may be a woven material. The rear face firstly has a layer of hypoallergenic adhesive, i.e., the side to be applied to the user's face, and a strippable protective backing sheet covering the adhesive back face. The adhesive is preferably a sterile, pressure-sensitive, hypoallergenic adhesive applied as a film on the back face of the devices. The adhesive is applied uniformly over the back surface with the exception of the area in which the tabs are located. The adhesive is of such a character that it is non-drying, is capable of remaining in a tacky state, and yet can be readily removed from a user's skin without injury to the user. Typically and as widely used silicone adhesives, such as those used for adhesive bandage strips, may be used in the present invention.

### Advantages

Such an anti-snoring device can be used to substantially reduce or stop snoring, and may be useful for people who have asthma since the device encourages breathing through the nose which is equipped to remove foreign particles from the air inhaled. Likewise the present invention aids people who suffer from such disorders as sleep apnoea. Sleep apnoea is a type of sleep disorder characterised by pauses in breathing or instances of shallow or infrequent breathing during sleep.

The present invention provides advantages of preventing snoring and the dry mouth often associated with snoring. The present invention provides for either a device which covers the lips or for supporting the lower jaw to keep it closed to stop snoring during sleep. The device is simple, comfortable and easy to use. The device can be placed either over your mouth or around your lips around the lower jaw to hold the jaw in place so that your tongue stays forward keeping your airways open allowing you to breathe naturally through your nose.

An air vent placed in the middle of the snore patch is provided in the event that the user may need to cough, sneeze or take a small breath through the mouth if needed. The air vent also provides peace of mind for people who are prone to anxiety attacks or suffer from the feeling of claustrophobia when the anti-snoring device is in place over the user's mouth. The air vent makes the user feel more secure when using the device for the first few times until they are comfortable using the device. There is a tab on each side of the snore patch for easy removal and disposal

It has been found that most people using this device can stop snoring immediately and tend to come to terms with breathing through the nose relatively quickly. The device has been designed to prevent or greatly reduce snoring and therefore is helpful in providing a good night sleep for not only the user but also their bed partner

### Variations

It will be realized that the foregoing has been given by way of illustrative example only and that all other modifications and variations as would be apparent to persons skilled in the art are deemed to fall within the broad scope and ambit of the invention as herein set forth.

In this specification, adjectives such as first and second, left and right, top and bottom, and the like may be used solely to distinguish one element or action from another element or action without necessarily requiring or implying any actual such relationship or order. Where the context permits, reference to an integer or a component or step (or the like) is not to be interpreted as being limited to only one of that integer, component, or step, but rather could be one or more of that integer, component, or step etc.

In the specification the term “comprising” shall be understood to have a broad meaning similar to the term “including” and will be understood to imply the inclusion of a stated integer or step or group of integers or steps but not the exclusion of any other integer or step or group of integers or steps. This definition also applies to variations on the term “comprising” such as “comprise” and “comprises”.

1. An anti-snoring device comprising:
a membrane shaped as an elongate oval having opposed sides and opposed arcuate ends, the membrane having a front and a rear face;
a hypoallergenic adhesive applied uniformly to the rear face of said membrane, and a backing sheet which covers the adhesive on the back of said membrane;
at least one tab formed at one of the arcuate ends of the membrane, such that the at least one tab allows the membrane to be easily manipulated between open and closed positions, the at least one tab being free from any adhesive; and
wherein the device is sized and configured to substantially cover a user's closed lips and an area of skin adjacent to each lip to hold the lips in a closed position.

2.-3. (canceled)

4. An anti-snoring device as claimed in claim 1, wherein the opposed sides comprise continuously convex curved opposed sides to generally conform to the contour of the user's upper and lower lips respectively and an area of skin adjacent to each lip.

5. An anti-snoring device as claimed in claim 4, wherein the membrane comprises a sheet of flexible, tear-resistant plastic, non-woven or woven fabric material.

6. (canceled)

7. An anti-snoring device as claimed in claim 5, wherein the membrane is manufactured from cotton, plastic or nylon, and the hypoallergenic adhesive is an epoxy resin.

8.-15. (canceled)

16. An anti-snoring device as claimed in claim 7, wherein said adhesive is applied to an area located adjacent the outer edges of said membrane.

17. (canceled)

18. An anti-snoring device as claimed in claim 16, further comprising a curved section located in one of the opposed sides to allow the device to be easily located within the contour or philtrum in between the top lip and the bottom of the nose of the user.

19.-22. (canceled)

23. An anti-snoring device for applying around the lower lip of a user comprising:
a flexible, elongate membrane having a perimeter, a generally uniform thickness and opposed faces;
a hypoallergenic adhesive applied uniformly to one face of said membrane, and a backing sheet which covers the adhesive;
a plurality of tabs extending around the perimeter of the membrane, such that the tabs allows the membrane to be easily manipulated between open and closed positions; and
wherein the device is sized and configured to substantially fit around the user's closed lower lip and an area of skin adjacent either end of the lips to hold the lower jaw in a closed position to inhibit mouth breathing.

24. An anti-snoring device as claimed in claim 23, wherein the flexible elongate membrane has an upper edge and a lower edge extending around the perimeter, wherein the lower edge conforms to the shape of the lower edge of the face of the user, including the area defined by the chin boss and extending to an area on either end of the lips to secure the lower jaw closed to prevent mouth breathing.

25. An anti-snoring device as claimed in claim 23, wherein the flexible elongate membrane has a upper edge and a lower edge extending around the perimeter, wherein the lower edge conforms to the shape of the lower edge of the face of the user, including the area defined by the chin boss and extending to an area spaced apart from either end of the lips to secure the lower jaw closed to inhibit or prevent mouth breathing.

26.-27. (canceled)

28. An anti-snoring device as claimed in claim 25, wherein the membrane comprises a sheet of flexible, tear-resistant plastic, woven or non-woven waterproof fabric material.

29. An anti-snoring device as claimed in claim 28, wherein the membrane is manufactured from cotton, plastic or nylon, and the hypoallergenic adhesive is an epoxy resin.

30. An anti-snoring device as claimed in claim 23, wherein the plurality of tabs extending around the perimeter of the membrane has a rear surface, the rear surface of the plurality of tabs is free from any hypoallergenic adhesive.

31. An anti-snoring device as claimed in claim 23, wherein the sheet of flexible material has two tabs, each tab formed at an end of the membrane.

32. An anti-snoring device as claimed in claim 31, wherein the tab or tabs on their rear surface are free from any hypoallergenic adhesive.

33. An anti-snoring device as claimed in claim 32, wherein the hypoallergenic adhesive is releasable and designed to be opened and closed without losing the ability to sufficiently hold the lower jaw in the closed position.

34. An anti-snoring device as claimed in claim 33, wherein the hypoallergenic adhesive is a sterile or non-sterile, pressure-sensitive, hypoallergenic adhesive and is applied as a film on the back face of the device.

35. An anti-snoring device as claimed in claim 34, wherein the hypoallergenic adhesive is of such a character that it is non-drying, is capable of remaining in a tacky state, and can be readily removed from a user's skin without injury to the user.

36. An anti-snoring device as claimed in claim 35, wherein the hypoallergenic adhesive is a silicone adhesive such as those used for adhesive bandage strips.

37. An anti-snoring device as claimed in claim 36, wherein the membrane is a transparent, multi-coloured or skin coloured tear resistant material, and the membrane has a design or picture(s) on one of the opposed sides of the tear resistant material.

38. An anti-snoring device as claimed in claim 37, wherein the adhesive is applied to an area located adjacent the perimeter of said membrane.
